(19) United States
(12) Patent Application Publication
(10) Pub. No.: US 2014/0155846 A1
(43) Pub. Date: Jun. 5, 2014

(54) Flatus Transporter

(71) Applicant: Sebastian Jack Choularton, Redondo Beach, CA (US)

(72) Inventor: Sebastian Jack Choularton, Redondo Beach, CA (US)

(21) Appl. No.: 13/691,455

(22) Filed: Nov. 30, 2012

(51) Int. Cl. A61M 1/00

(52) U.S. Cl. CPC A61M 1/0019
USPC 604/317

(57) ABSTRACT

The present flatus transporter provides a novel and unique device for effectively capturing gaseous flatus from the anus region and permitting the user to transport the captured flatus to another location, then release the gaseous flatus in a controlled and targeted manner. The flatus transporter includes a cupped member connected to a resilient hollow bulb through a port that permits gaseous flatus to travel into the bulb from the cupped member. The user applies the cupped member over the anus, which draws in the gaseous flatus under the vacuum created by releasing the squeezed bulb. The captured gaseous flatus can be pushed out of the bulb with great velocity and directional control by squeezing the bulb. The bulb effectively stores the flatus for long periods so that the user may choose the time and place of expulsion.

### RELATED APPLICATION DATA

This application claims the priority date of provisional application No. 61/566,538 filed on Dec. 2, 2011.

### BACKGROUND

The present device relates generally to intestinal discharge control products, and more particularly to the capture and transport of gaseous flatus.

The uncontrolled release of flatulence is a persistent issue, which people have try regulate with limited success. A number of methods are employed for this purpose, including burning candles or matches, use of incense, opening windows, switching on fans, and the like. These methods attempt to remove the flatus so that it cannot be smelled; however, these methods generally permit the flatus to escape to the atmosphere in an uncontrolled manner. Other devices employ a user-donned filtering pad, with activated carbon or a similar absorptive media. These devices require the user to wear the pad near the anal region at all times during which flatulence may be expected. This could be a costly and an uncomfortable means to eliminate errant flatus, as the pads are consumed on a daily basis and may create an uncomfortable protuberance.

What is needed is a device that directly targets the anal source of flatus, capturing the gaseous emissions before being permitted to disperse at the point of release. What is also needed is a device that can transport the captured flatus within a portable means, such that the flatus can transported to a desired release location. What is further needed is a device that permits the user to conveniently release the captured flatus, in a targeted manner, to the desired release target.

### SUMMARY

The present device and method provides a novel and unique way to easily capture flatus as it is expelled from the anus. As the anus is not readily visible and the opportunity to capture flatulence is fleeting, the present device permits the user to quickly locate the device over the anus without undue adjustment. Furthermore, the present device enables the expedited absorption and retainment of flatus before localized dispersal. Yet another unique feature of the present device includes the ability to transport the captured flatus and targeted release of the flatus to a desired target location.

The present device is a flatus transporter, which includes primarily a hollow, compressible bulb gaseously connected with an externally located cupped member. In particular, the bulb has a compressible wall defining a hollow interior and a port to permit ingress and egress of gaseous flatus. The cupped member is generally cup-shaped with a bottom wall and annular side wall, with a primary opening with a rim. An aperture is preferably formed through the bottom wall, but may be formed elsewhere. The aperture is configured to be in gaseous communication with the port. Optionally, the side wall of the cupped member may be substantially cylindrical nearest to the primary opening, the wall transitioning to a funnel shape nearest to the aperture, the funnel shape configured to direct the flow of gaseous flatus into the aperture under the vacuum effect.

During use, the hollow interior is configured to be collapsed by squeezing the compressible wall. The rim of the cupped member is configured to be applied to the buttocks, such that the rim surrounds the anus when expelling flatus. Ideally, the user should squeeze the bulb prior to release of the flatus. When the squeezing force on the bulb is released, the bulb is configured to create a vacuum effect within the cupped member through resilient expansion of the bulb. Essentially, the expanding bulb draws in gas through the port to fill the hollow interior. Thus, because the cupped member substantially encloses the anus region, gaseous flatus is drawn under vacuum into the primary opening, through the aperture, and through the port. Thereafter, the flatus is stored within the hollow interior until selective evacuation through the port by again squeezing the compressible wall

Optionally, a stable bottom can be formed on the exterior of the compressible wall of the bulb, such that the stable bottom is configured to rest upon a support surface and prevent the bulb from rolling.

As an optional feature, the port may be controlled or restricted by a valve. In particular, the port may be a resilient slit valve formed by piercing a slit through a membrane portion of the compressible wall. There can be formed a single slit, cross slits, or multiple intersecting slits. The resilient slit valve controllably inhibits ingress and egress of gas when the slit is closed. The slit is configured to open or separate at the seam of the slit when the bulb is being compressed or resiliently expanded to permit gas flow through the resilient slit valve.

Optionally, the cupped member may further comprise a hollow tenon protruding from the wall with the aperture formed at a tenon terminus. The interior of the hollow tenon provides gaseous communication between the primary opening and the aperture. During use, the hollow tenon is configured to be inserted through the resilient slit valve, with the resilient slit valve being forced open and tightly gripping the outer surface of the hollow tenon. The hollow tenon can be made of various materials, but preferably is molded of the same plastic or rubber material the cupped member is molded from, and may be molded together with the cupped member.

Again optionally, a hollow stem may protrude from the compressible wall of the bulb, where the slit valve or other valve member is formed at the terminus of the stem. The hollow stem is configured to provide gaseous communication between the slit valve and the hollow interior. Another variation of the port valve member, is a hole formed through a thin resilient membrane, the thin resilient membrane restricting the ingress and egress of gas. The thin resilient membrane may be flat with a centrally located hole. The thin resilient membrane may also be radially pleated, such that the radial pleats provide additional material to facilitate the widening of the hole when the tenon is inserted, yet the pleats are able to resilient return to an original position where the hole is of a minimized size.

In an alternate embodiment, a hose fitting provides a removable connection between the bulb and the cupped member. As is standard in industry, the hose fitting generally has a barbed connector and a threaded connector, with a gas passage formed through it. In this example, the threaded connector is configured to be threaded into the aperture of the cupped member, while the barbed connector is configured to be inserted into the port of the bulb in frictional engagement. However, other connections are applicable, including a hose fitting-type connector, with each end glued into its respective port or aperture. The hose fitting is configured to provide gaseous communication between the aperture and the port and, optionally, to permit quick detachment of the cupped member and the bulb by withdrawal of the barbed connector from the port if the connection is not glued.

In yet another alternate embodiment, flatus transporter for the storage and transport of gaseous flatus is provided. This flatus transporter primarily includes a bulb with a compressible wall defining a hollow interior, a cupped member extending from the bulb with a wall that defines an open-ended chamber with a rim, and a port formed between the bulb and the cupped member to provide a gas connection between the open-ended chamber and the hollow interior to permit ingress and egress of gaseous flatus through the port. Optionally, the wall of the cupped member is substantially cylindrical nearest to the rim, and the wall transitions to a funnel shape nearest to the port. The funnel shape is configured to direct the flow of gaseous flatus into the port under vacuum.

Additionally, a method of capturing gaseous flatus is provided. The first step includes providing a flatus transporter with a cup-shaped member extending from an exterior of a resiliently compressible bulb, with a port creating a gas connection between an open chamber of the cup-shaped member and a hollow interior of the resiliently compressible bulb. Next, the user squeezes the resiliently compressible bulb to collapse the hollow interior. Then, the user applies the cup-shaped member to the buttocks to substantially enclose the open chamber, where the buttocks blocks the open chamber. The user then positions the cup-shaped member such that the open chamber is positioned so that it covers and surrounds the anus. Afterwards, the user releases the resiliently compressible bulb near the time of expulsion of flatus. The device creates a vacuum effect within the open chamber through the resilient expansion of the bulb, which draws in gaseous flatus into the open chamber and through the port. Finally, the gaseous flatus is stored within the hollow interior.

Optionally, the method may include further steps. The flatus transporter may be transported to a location away from the buttocks. The user again squeezes the resiliently compressible bulb to collapse the hollow interior to expel a jet of the gaseous flatus to the location. Again optionally, the user may direct the port towards a desired target location after transporting the flatus transporter and before squeezing the resiliently compressible bulb, such that the jet of the gaseous flatus is delivered to the desired target location.

### BRIEF DESCRIPTION OF THE SEVERAL VIEWS OF THE DRAWINGS

FIG. 1A is a top planar view of an example embodiment of a flatus transporter;

FIG. 1B is a side planar view of the flatus transporter of FIG. 1A;

FIG. 1C is an perspective view of the flatus transporter of FIG. 1A-B;

FIG. 2 is an exploded side planar view of the flatus transporter, with hidden lines showing the interior features;

FIGS. 3A-B are side views showing the present flatus transporter in use and against the buttocks;

FIG. 3C is a sectional view of the flatus transporter, with the section taken through the central axis, showing the gas flow and storage within;

FIG. 3D is a side view of the flatus transporter which has been transported to a target location and the flatus released at that location;

FIG. 4 is an exploded perspective view of an alternate embodiment of the flatus transporter; and

FIGS. 5A-B are top planar views of the various valve arrangements for the flatus transporter.

### LIST OF REFERENCE NUMERALS OF FIRST-PREFERRED EMBODIMENT

flatus transporter 10
bulb 12
compressible wall 14
hollow interior 16
port 18
cupped member 20
open-ended chamber 22
primary opening 24
rim 26
cylindrical wall 28
bottom wall 30
funnel portion 32
aperture 34
wall 36
hose fitting 38
barbed connector 40
threaded connector 42
stable bottom 44
resilient slit valve 46
slit 48
membrane 50
hole 52
pleat 54
membrane 56
hollow tenon 58
aperture 60
terminus 62
hollow stem 64
terminus 66
ring 68
port 70
membrane valve member 72
plug 74
squeezing force F
resilient force R
user 200
buttocks 202
gaseous flatus 204
flatus jet 206
support 300
target location 400
vacuum flow 500

### DESCRIPTION OF THE PREFERRED EMBODIMENTS

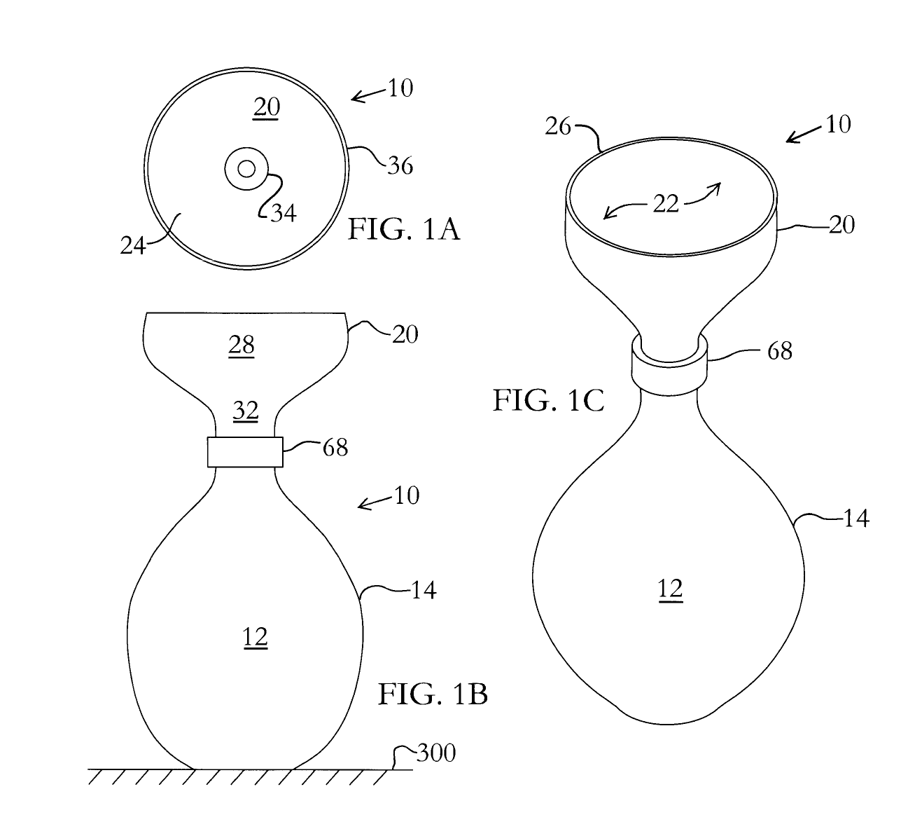

Looking first at FIGS. 1A-C, the flatus transporter (10) is generally shown in several views. The cupped member (20) is generally shaped like a cup or bowl, with a rim (26) and an open-ended chamber (22) surrounded on all but one side by a wall (36). Although the cupped member (20) is described as “cup-shaped,” the shape does not necessarily need to be circular. Other shapes that maintain the function of the cupped member (20) are possible, such as shapes that facilitate greater flow characteristics or shapes that create a better seal against the buttocks (202). In this example embodiment, the cupped member (20) has a primary opening (24) which is designed to maximize the area around the anus covered while the flatus transporter is applied to the buttocks (202). The cupped member (20) is shown with a cylindrical wall (28) portion that transitions into a bottom wall (30) that can be shaped to create a funnel portion (32). As will be described in detail below, the funnel portion (32) facilitates vacuum flow within the open-ended chamber (22) so that the entire body of the gaseous flatus (204) can more easily be drawn in. On the bottom wall (30), and in this case at the bottom of the funnel portion (32), is an aperture (34). The rim (26) can be constructed of a soft material, such as foam, rubber, or the like, to provide cushioning or a better seal. Portions of the cupped member (20) or the entirety can be made of hard plastic, such as HDPE or hard vinyl.

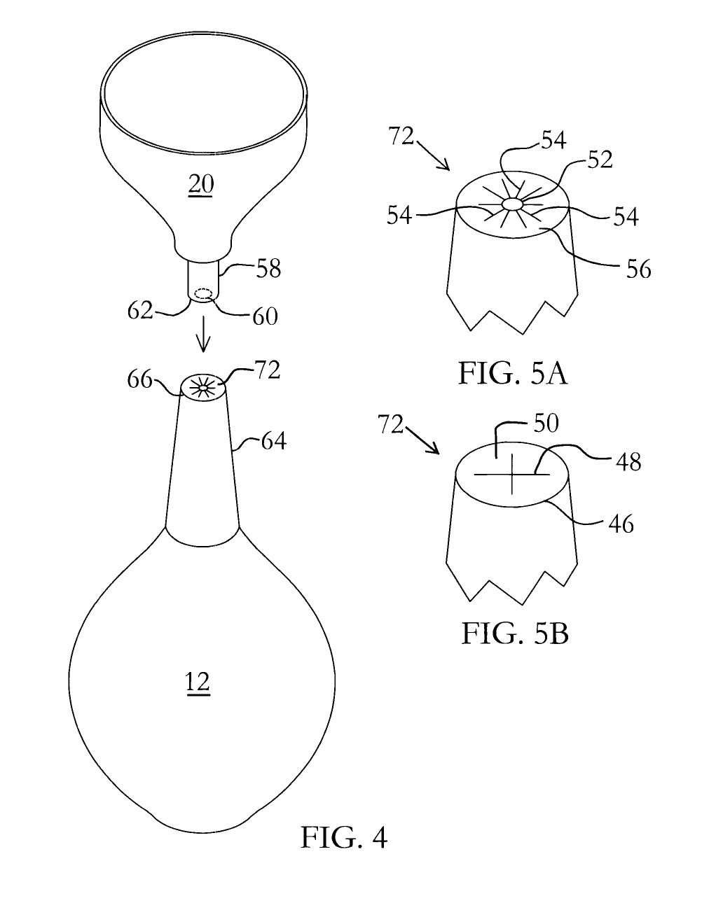

The bulb (12) is generally bulbous and hollow, with a compressible wall (14) and a hollow interior (16). The bulb (12) is made of a soft but firm material that can be repeatedly squeezed and manipulated without permanent deformation or tearing, such as a rubber compound, flexible plastic material, or the like. The bottom of the bulb (12) or any other part of the compressible wall (14) can be made with a slight concave portion or a flat portion to create a stable bottom (44), so that the bulb (12) alone or attached with the cupped member (20) can be placed upon an support surface (300) so that the bulb (12) will be prevented from rolling, as is shown in FIG. 4.

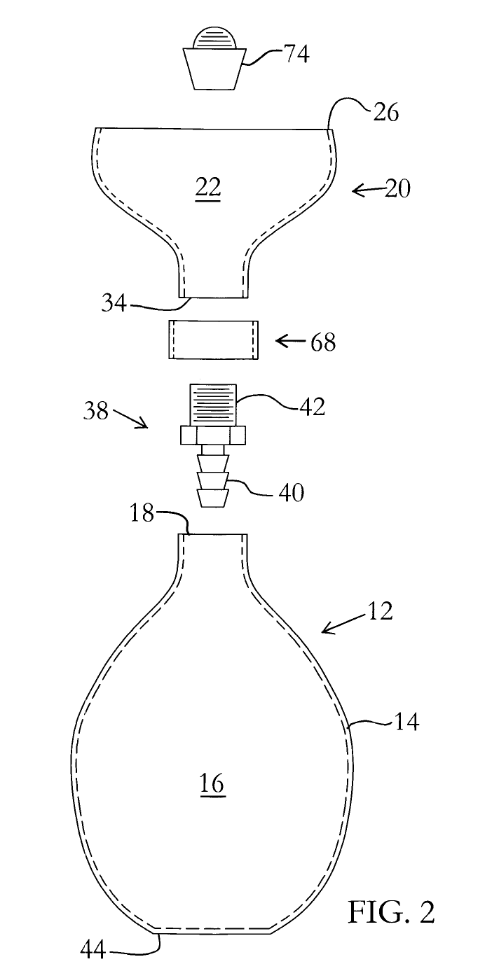

In this example embodiment, looking also at FIG. 2, the cupped member (20) is manufactured separately from the bulb (12). The cupped member (20) is fastened atop the bulb (12), such that the aperture (34) of the cupped member (20) is aligned with the port (18) of the bulb (12). The aperture (34) and the port (18) may be directly connected through gluing, heat sealing, sonic welding, or similar process, so that a gas may hermetically travel though the connection from the open-ended chamber (22) to the hollow interior (16), or vice versa. FIG. 2 shows an exploded view of the flatus transporter (10), where the hidden lines indicate the interior wall of the cupped member (20) and the bulb (12). The interior wall of the cupped member (20) converges to form the aperture (34). Likewise, the interior wall of the bulb (12) converges to form the port (18). Either the port (18) or aperture (34) can be plugged with plug (74), which is a common plug or stopper made form cork, rubber, or the like, which would prevent the gaseous flatus from escaping the hollow interior (16).

Alternately, the aperture (34) may be connected to the port (18) through a connector, in this case a hose fitting (38). The hose fitting (38) in this example is a standard industry part, and has a barbed connector (40) and a threaded connector (42) with a gas passage extending though the hose fitting (38). Other standard industry hose fittings (38) can be used, including one with two threaded or two barbed connectors. The threaded connector (42) can be frictionally pushed into the aperture (34) or threaded in if threads are molded on the internal diameter of the aperture (34). The barbed connector (40) is pushed into frictional engagement within the port (18) of the bulb (12). The hose fitting (38) may glued to one or both of the cupped member (20) and the bulb (12). Alternatively, the cupped member (20) may be detachable from the bulb (12) by leaving the barbed connector (40) unglued from the port (18) so that the cupped member (20) with the hose fitting (38) can be removed as a unit from the bulb (12). A ring (68) may be slid over the joint between the cupped member (20) and the bulb (12) to hide the seam and hose fitting (38) or to provide an additional mechanical connection, where the ring (68) provides a glued or welded connection between the bulb (12) and the cupped member (20). However, the ring (68) may also serve just a decorative purpose.

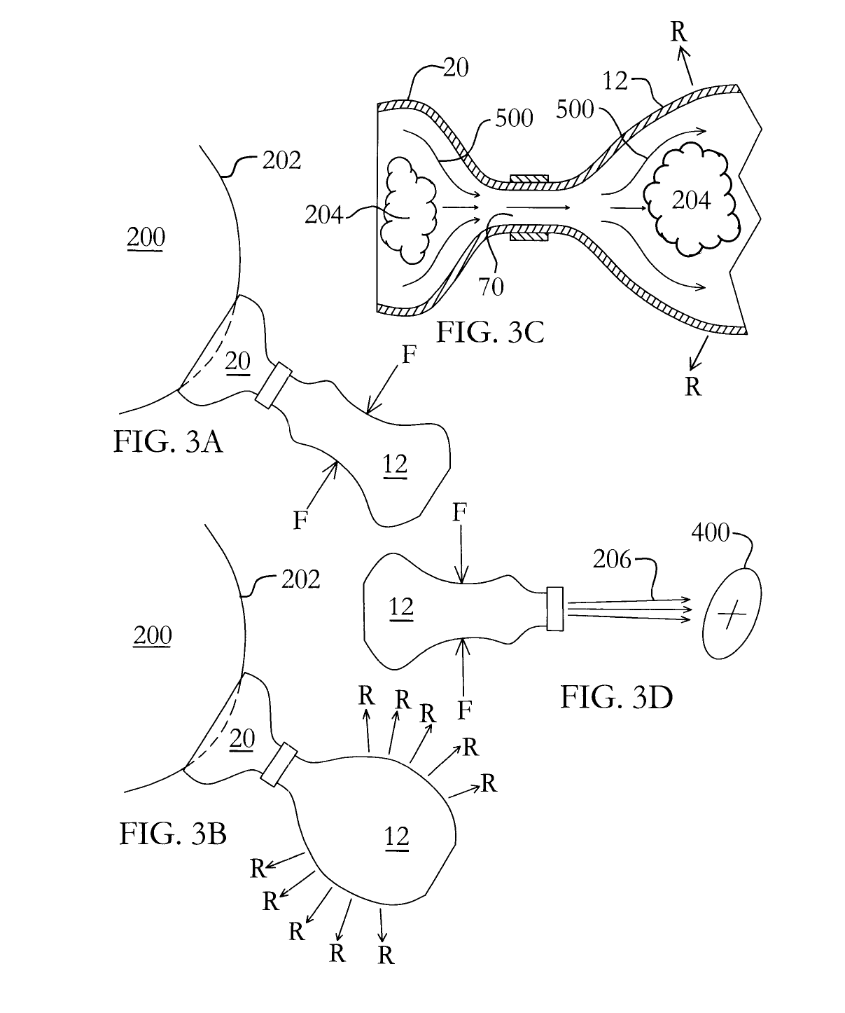

Now, turning to FIGS. 3A-D, the flatus transporter (10) is shown in use. Upon a sensation of impending flatulence, the user (200) manually applies a squeezing force (F) to the exterior of the compressible wall (14) of the bulb (12) to deform the bulb (12) and decrease the volume of the hollow interior (16). The rim (26) of the cupped member (20) is applied to the buttocks (202) to create a seal against the buttocks (202), which substantially encloses the open-ended chamber (22). The seal does not need to be air-tight or perfect, just sufficient to facilitate the capture of a significant quantity of flatus through vacuum flow. The user (200) can apply the cupped member (20) to the buttocks (202) before or after squeezing the bulb (12). Ideally, the bulb (12) should be squeezed before expelling the flatus (204) so the suction function is at the ready. However, the user (200) may still benefit after expelling the flatus (204) if the cupped member (20) and the squeezed bulb (12) is applied quickly after the fact. The squeezing force (F) is represented by the opposing arrows, which schematically represents the force as applied by the user's (200) hand (not shown). For example, the user (200) may squeeze the bulb (12) between the four fingers and the opposed thumb.

Ideally, after or during the release of the gaseous flatus (204), the squeezing force (F) is released to permit the resilient force (R). The resilient force (R) is represented with arrows pointed in a radially outward direction, and is produced by the spring force of the resilient bulb material. The user (200) would still be holding the flatus transporter (10), but would not be squeezing the bulb (12) in any significant way. The user (200) could also control the rate of bulb (12) expansion by regulating the release of the bulb (12), to coincide with longer periods of flatulence. The action of the expanding bulb (12) can be more clearly seen in FIG. 3C. The user (200) releases the gaseous flatus (204) into the open-ended chamber (22) of the cupped member (20). The user (200) releases the squeezing pressure on the bulb (12), which expands towards its original shape. As the bulb (12) expands, it draws in the gaseous flatus (204) held within the cupped member (20) through the port (70) and into the hollow interior of the bulb (12). The vacuum flow (500) created by the expanding bulb (12) is generally indicated by the arrows leading through the port (70). In this example embodiment, the bulb (12) and the cupped member (20) are molded together. One method of molding the two together would include molding the assembly in two symmetric halves and adhering or welding the halves together. In this way, the port (70) is just a restricted passageway between the open-ended chamber (22) and the hollow interior (16).

FIG. 3D shows an embodiment of the flatus transporter (10) that permits the removal of the cupped member (20) from the bulb (12), so that the bulb (12) with the contained gaseous flatus (204) can be transported to a desired location for release. In particular, the bulb (12) is squeezed by applying a squeezing force (F), as indicated by the opposed arrows. This squeezing causes the gaseous flatus (204) to rush out from the hollow interior (16) of the bulb (12) in a flatus jet (206), which is easily directed with precision to a target location (400) of the user's (200) choosing. The speed of the flatus jet (206) and the distance it travels can be regulated by varying the force and speed by which the bulb (12) is squeezed.

An alternate embodiment of the flatus transporter (10) can be seen in FIG. 4, which shows a version in which the cupped member (20) is detachable from the bulb (12). The cupped member (20) includes a hollow tenon (58) with an aperture (60) at the terminus (62). The bulb (12) includes a hollow stem (64) that extends from the body of the bulb (12) with a membrane valve member (72) formed at the terminus (66). The hollow stem (64) increases the length of the passageway through which the flatus (204) must travel to be expelled, which can enhance the jet effect and directional accuracy. Two types of membrane valve members (72) can be seen more clearly when viewing FIGS. 5A-B. FIG. 5A shows a resilient membrane (56) with pleats (54) folded radially about a central hole (52). The hollow tenon (58) is configured to be pressed into the hollow stem (64) through the membrane valve member (72). In the case of the pleated member, the hollow tenon (58) is pushed into the hole (52), where the pleats (54) permit the hole (52) to expand to a larger size with the resilient membrane (56) stretching to tightly grip the hollow tenon (58). In this way, the membrane valve member (72) mates with the hollow tenon (58) to form a connection by a gripping engagement. Furthermore, the variable-sized hole (52) permits the removal of the cupped member (20) with the hole (52) immediately springing back to its minimal diameter so that the gaseous flatus (204) is prevented from escape during long periods of storage. Optionally, the cupped member (20) may just have an aperture (34), as shown in FIG. 2, and not the hollow tenon (58). With this arrangement, the aperture (34) is sized larger than the hollow stem (64), so that the hollow stem (64) can be inserted into the aperture (34) to create a frictional engagement.

The resilient slit valve (46) of FIG. 5B similarly minimizes the opening to prevent flatus (204) from escape. The resilient slit valve (46) has a resilient membrane (50) with one or more slits (48) pierced through the membrane (50). The slits (48) are shown in a cross pattern in this example, but other patterns are compatible. The slits (48) are normally closed with the membrane (50) in a flat state. As vacuum flow in drawn in or pressure flow expelled out by respectively releasing and squeezing the bulb (12), the pressure of the gas flow causes the slit (48) to separate and the membrane (50) to slightly deform. In this way, the gaseous flatus (204) can travel through the slit valve (48) when open. Further, the slit valve (46) can receive the hollow tenon (58), where the hollow tenon (58) is pushed through the slits (48) with the membrane (50) tightly gripping the hollow tenon (58).

1. A flatus device for transporting gaseous flatus from a user's anus, comprising:
a bulb with a compressible wall defining a hollow interior and a port to permit ingress and egress of gaseous flatus; and
a cupped member with a primary opening defining a rim and an aperture formed through a wall, the aperture configured to be in gaseous communication with the port the cupped member being directly connected to the bulb;
wherein the hollow interior is configured to be collapsed by squeezing the compressible wall, and the rim of the cupped member is configured to be applied such that the rim surrounds the user's anus when expelling flatus;
and wherein the bulb is configured to create a vacuum effect within the cupped member through resilient expansion of the bulb, such that gaseous flatus is drawn into the primary opening, through the aperture and through the port, thereafter to be stored within the hollow interior until selective evacuation through the port by again squeezing the compressible wall.

2. The flatus device of claim 1 wherein a stable bottom is formed externally on the compressible wall of the bulb, such that the stable bottom is configured to rest upon a support surface and prevent the bulb from rolling.

3. The flatus device of claim 1 wherein the port is a resilient slit valve formed by piercing a slit through a membrane portion of the compressible wall, the resilient slit inhibiting ingress and egress of gas when the slit is closed, the slit configured to open when the bulb is being compressed or resiliently expanded to permit gas flow through the resilient slit valve.

4. The flatus device of claim 3 wherein the cupped member further comprises a hollow tenon protruding from the wall with the aperture formed at a tenon terminus, the hollow tenon providing gaseous communication between the primary opening and the aperture;
wherein the hollow tenon is configured to be inserted through the resilient slit valve with the resilient slit valve being forced open and tightly gripping an outer surface of the hollow tenon.

5. The flatus device of claim 4 wherein a hollow stem protrudes from the compressible wall of the bulb, the slit valve being formed at a stem terminus with the hollow stem providing gaseous communication between the slit valve and the hollow interior.

6. The flatus device of claim 1 wherein the port is a thin resilient membrane comprising a hole formed through the thin resilient membrane, the thin resilient membrane restricting the ingress and egress of gas.

7. The flatus device of claim 6 wherein the cupped member further comprises a hollow tenon protruding from the wall with the aperture formed at a tenon terminus, the hollow tenon providing gaseous communication between the primary opening and the aperture;
wherein the hollow tenon is configured to be inserted through the hole of the thin resilient membrane with the hole stretched to tightly gripping an outer surface of the hollow tenon.

8. The flatus device of claim 1 wherein a hose fitting provides a removable connection between the bulb and the cupped member, the hose fitting comprising a barbed connector and a threaded connector, the threaded connector configured to be threaded into the aperture of the cupped member, the barbed connector configured to be inserted into the port of the bulb in frictional engagement;
wherein the hose fitting is configured to provide gaseous communication between the aperture and the port and to permit quick detachment of the cupped member and the bulb by withdrawal of the barbed connector from the port.

9. The flatus device of claim 1 wherein the wall of the cupped member is substantially cylindrical nearest to the primary opening, the wall transitioning to a funnel shape nearest to the aperture, the funnel shape configured to direct the flow of gaseous flatus into the aperture under the vacuum effect.

10. A flatus transporter for the storage and transport of gaseous flatus, comprising:
a bulb with a compressible wall defining a hollow interior;
a cupped member extending directly from the bulb and having a wall that defines an open-ended chamber with a rim; and
a port formed between the bulb and the cupped member, the port providing a gaseous connection between the open-ended chamber and the hollow interior to permit ingress and egress of gaseous flatus through the port;
wherein the hollow interior is configured to be collapsed by squeezing the compressible wall, and the rim of the cupped member is configured to be applied to a user's buttocks such that the rim surrounds a user's anus when expelling flatus;
and wherein the bulb is configured to create a vacuum effect within the open-ended chamber through resilient expansion of the bulb, such that gaseous flatus is drawn into the open-ended chamber and through the port, thereafter to be stored within the hollow interior until selective evacuation through the port by again squeezing the compressible wall.

11. The flatus transporter of claim 10 wherein a stable bottom is formed externally on the compressible wall of the bulb, such that the stable bottom is configured to rest upon a support surface and prevent the bulb from rolling.

12. The flatus transporter of claim 10 wherein the wall of the cupped member is substantially cylindrical nearest to the rim, the wall transitioning to a funnel shape nearest to the port, the funnel shape configured to direct the flow of gaseous flatus into the port under the vacuum effect.

13. A method of capturing gaseous flatus, comprising the steps of:
providing a flatus transporter having a cup-shaped member directly extending from an exterior of a resiliently compressible bulb, with a port creating a gaseous connection between an open chamber of the cup-shaped member and a hollow interior of the resiliently compressible bulb;
squeezing the resiliently compressible bulb to collapse the hollow interior;
applying the cup-shaped member to a user's buttocks to substantially enclose the open chamber;
positioning the cup-shaped member such that the open chamber is positioned above a user's anus and surrounding the anus;
releasing the resiliently compressible bulb near the time of expulsion of flatus;
creating a vacuum effect within the open chamber through the resilient expansion of the bulb;
drawing gaseous flatus into the open chamber and through the port; and
storing gaseous flatus within the hollow interior.

14. The method of capturing gaseous flatus of claim 13, further comprising the steps of:
transporting the flatus transporter to a target location away from the buttocks;
directing the port towards the target location;
squeezing the resiliently compressible bulb to collapse the hollow interior; and
expelling a jet of the gaseous flatus to the target location.

15. The method of capturing gaseous flatus of claim 14, further comprising the steps of:
inserting a plug in the port after the step of drawing gaseous flatus into the open chamber and through the port; and
removing the plug before the step of directing the port towards the target location.
